(19) United States
(12) Patent Application Publication
(10) Pub. No.: US 2016/0166811 A1
(43) Pub. Date: Jun. 16, 2016

(54) THROMBECTOMY AND BALLOON ANGIOPLASTY/STENTING DEVICE

(71) Applicant: Khaldoon Alaswad, Detroit, MI (US)

(72) Inventor: Khaldoon Alaswad, Detroit, MI (US)

(21) Appl. No.: 14/748,346

(22) Filed: Jun. 24, 2015

Related U.S. Application Data
(63) Continuation of application No. 12/473,063, filed on May 27, 2009, now abandoned.

(51) Int. Cl. A61M 25/10; A61M 25/00; A61F 2/958; A61M 25/09

(52) U.S. Cl. CPC A61M 25/104; A61M 25/09; A61M 25/1011; A61M 25/0026; A61M 25/007; A61M 25/1002; A61F 2/958; A61M 2025/1052; A61B 2017/22079

(57) ABSTRACT

A thrombectomy and balloon angioplasty catheter for the removal of blood clots and debris from a blood vessel and/or for the dilation of a narrowed or completely closed segment in the blood vessel is disclosed. The catheter includes a shaft having a proximal end and a distal end, an angioplasty balloon mounted to the shaft adjacent the distal end of the shaft, and an occlusion balloon mounted to the shaft at a location proximally spaced from the angioplasty balloon. A guide wire lumen is arranged on the shaft. The guide wire lumen extends from a first position adjacent the distal end of the shaft to a second position proximal the angioplasty balloon and distal the proximal end of the shaft. The shaft includes an angioplasty balloon inflation lumen in fluid communication with the proximal end of the shaft and an interior space of the angioplasty balloon, an occlusion balloon inflation lumen in fluid communication with the proximal end of the shaft and an interior space of the occlusion balloon, and a thrombectomy lumen in fluid communication with the proximal end of the shaft and a thrombectomy suction port located between the angioplasty balloon and the occlusion balloon.

### CROSS-REFERENCES TO RELATED APPLICATIONS

This application is a continuation of U.S. patent application Ser. No. 12/473,063 filed May 27, 2009.

### STATEMENT REGARDING FEDERALLY SPONSORED RESEARCH

Not Applicable.

### BACKGROUND OF THE INVENTION

1. Field of the Invention

The invention relates to medical devices for the removal of the blood clots and debris from a blood vessel and/or for the dilation of a narrowed or completely closed segment in the blood vessel.

2. Description of the Related Art

Atherosclerosis involves a buildup of plaque that narrows a blood vessel making it more difficult for blood to flow through the vessel. Although atherosclerosis commonly develops in the arteries of the heart, it can strike any blood vessel, such as those feeding the brain, legs, or the kidneys. Atherosclerosis reduces the flow of oxygen-rich blood to these organs.

Balloon angioplasty (also called “percutaneous transluminal angioplasty”-PTA) is a technique performed to increase the size of the opening within the blood vessel by dilating a narrowed or completely closed segment in the blood vessel. The term “percutaneous transluminal coronary angioplasty” (PTCA) is used when the treatment is more specifically employed in vessels of the heart.

During balloon angioplasty, the physician will use a guide catheter or any other kind of catheter to engage main blood vessel, then the physician uses a guide wire to cross the narrowed or blocked area down stream in the blood vessel, and the physician threads a narrow balloon-tipped catheter over the guide wire and inside the guide catheter to the site of obstruction in the affected blood vessel. Once the balloon is in place, the physician inflates and deflates the balloon to eliminate the blood vessel obstruction. The deflated balloon is then removed. The goals of catheter intervention are to establish blood flow in the occluded or narrowed blood vessel, and to achieve optimal tissue perfusion.

Although balloon angioplasty is often successful in enlarging the opening of an obstructed blood vessel, an affected blood vessel may close again. When this happens, the physician may choose to perform another angioplasty and place a stent within the blood vessel. A stent is a cylindrical mesh wire to scaffold a narrowed or completely closed segment in a blood vessel and keep it open. Stent placement is much the same as angioplasty, except that the stent is crimped over the deflated balloon on the catheter that is threaded into the femoral artery. When the physician has inserted the catheter to the site of the obstruction, the balloon is inflated, causing the stent to expand. The physician then removes the balloon, leaving the expanded stent in place and allowing blood to once again flow freely through the blood vessel. Some stents are self expanding without balloon inflation. Stents can be placed in virtually any blood vessel of the body where blood flow is especially vital, including those of the heart, kidneys, and the carotid arteries which supply blood to the brain.

During balloon angioplasty and/or stenting, there is a risk that debris may be released from the occluded or narrowed area in the blood vessel. The debris could travel to plug the smaller branches or the microcirculation downstream from the stenosis. It has been reported that distal embolization occurs in up to 15% of angioplasty for heart attack cases, and is associated with increased 5 year mortality; 44% versus 9% in those without distal embolization.

Devices have been proposed to address the problem of debris being released from the occluded or narrowed area in the blood vessel during balloon angioplasty and/or stenting. For example, one device cuts debris into small sizes that do not occlude blood vessels. However, this device can be unduly complicated. Other devices include a filter for catching debris from balloon angioplasty and/or stenting. Of course, the filter adds additional complexity to the device and does not protect all side branches downstream from the angioplasty site. Another device (see, e.g., U.S. Pat. No. 6,485,500) uses a number of catheters wherein a first catheter is advanced over a guide wire and a second catheter is advanced over the first catheter such that occlusion balloons are located on opposite sides of the occluded or narrowed area in the blood vessel during balloon angioplasty and/or stenting thereby containing any debris released from the occluded or narrowed area in the blood vessel during balloon angioplasty and/or stenting. However, this device requires the use of multiple occlusion balloons, and it can be difficult to control in the surgical field in that the first catheter uses a guide wire lumen that extends the entire length of the catheter and the second catheter has another full length lumen such that the second catheter must be advanced over much of the length of the first catheter.

Therefore, while these known devices may be suitable for addressing the problem of distal embolization during balloon angioplasty and/or stenting, there still exists a need for an improved device and method for opening blood vessels without distal embolization.

### SUMMARY OF THE INVENTION

The present invention addresses the foregoing needs by providing a thrombectomy and balloon angioplasty catheter for the removal of blood clots and debris from a blood vessel and/or for the dilation of a narrowed or completely closed segment in the blood vessel. The catheter includes a shaft having a proximal end and a distal end, an angioplasty balloon mounted to the shaft adjacent the distal end of the shaft, and an occlusion balloon mounted to the shaft at a location proximally spaced from the angioplasty balloon. A guide wire lumen is arranged on the shaft. The guide wire lumen extends from a first position adjacent the distal end of the shaft to a second position proximal the angioplasty balloon and distal the proximal end of the shaft. The shaft includes an angioplasty balloon inflation lumen in fluid communication with the proximal end of the shaft and an interior space of the angioplasty balloon, an occlusion balloon inflation lumen in fluid communication with the proximal end of the shaft and an interior space of the occlusion balloon, and a thrombectomy lumen in fluid communication with the proximal end of the shaft and a thrombectomy suction port located between the angioplasty balloon and the occlusion balloon. The catheter can include an expandable stent arranged over the angioplasty balloon.

In one version of the catheter, the guide wire lumen extends from the first position adjacent the distal end of the shaft to a second position proximal the occlusion balloon. In another version of the catheter, the guide wire lumen extends from the first position adjacent the distal end of the shaft to a second position distal the occlusion balloon. In a preferred form of the catheter, no balloons are located distal the angioplasty balloon, and the balloons are non-perforated.

The catheter can further include means for inflating the angioplasty balloon such that a distal diameter of the angioplasty balloon reaches its maximum dimension before a proximal diameter of the angioplasty balloon reaches its maximum dimension. The means for inflating the angioplasty balloon can include an inflation port in fluid communication with the angioplasty balloon inflation lumen wherein the inflation port is located adjacent a distal end of the interior space of the angioplasty balloon. The inflation port can include a one way check valve that allows fluid flow into the interior space of the angioplasty balloon. The means for inflating the angioplasty balloon can be an angioplasty balloon comprising a more compliant material adjacent the distal end of the angioplasty balloon and a less complaint material adjacent the proximal end of the angioplasty balloon.

The catheter can further include means for deflating the angioplasty balloon such that the distal diameter of the angioplasty balloon reaches its relaxed position after the proximal diameter of the angioplasty balloon reaches its relaxed position. The means for deflating the angioplasty balloon can include a deflation port in fluid communication with the angioplasty balloon inflation lumen wherein the deflation port is located adjacent a proximal end of the interior space of the angioplasty balloon. The deflation port can include a one way check valve that allows fluid flow out of the interior space of the angioplasty balloon.

In this version of the catheter, the angioplasty balloon inflates gradually and not cylindrically, i.e. the balloon will start inflating from distal to proximal, and stay bigger at the distal end compared to the proximal end. This squeezes the debris back into the thrombectomy catheter. When the angioplasty balloon is deflated; it will deflate from proximal to distal allowing the aspiration of the debris before the distal end is fully deflated. Thus, the distal end of the angioplasty balloon functions as a distal protection balloon.

The catheter can include an angioplasty balloon inflation port located at the proximal end of the shaft wherein the angioplasty balloon inflation port is in fluid communication with the angioplasty balloon inflation lumen, an occlusion balloon inflation port located at the proximal end of the shaft wherein the occlusion balloon inflation port is in fluid communication with the occlusion balloon inflation lumen, and a thrombectomy exit port located at the proximal end of the shaft wherein the thrombectomy exit port is in fluid communication with the thrombectomy lumen. The angioplasty balloon inflation port, the occlusion balloon inflation port, and the thrombectomy exit port can be integrally arranged in a manifold that removably engages the proximal end of the shaft.

It is therefore an advantage of the invention to provide a thrombectomy and balloon angioplasty/stenting device wherein the device combines protection, thrombectomy, and therapy in one easy to use device.

It is another advantage of the invention to provide a thrombectomy and balloon angioplasty/stenting device wherein the device is compatible with the current coronary, bypass, and peripheral vascular intervention usual tools, and there is no need for special guide catheters or guide wires.

It is yet another advantage of the invention to provide a thrombectomy and balloon angioplasty/stenting device wherein the device protects the main branch and side branches in the native circulation as opposed to distal protection. This protects the entire myocardium at risk for distal embolization. This device is better than the distal protection in the native circulation, which has been shown to be not effective in the native coronary circulation in clinical trials.

It is still another advantage of the invention to provide a thrombectomy and balloon angioplasty/stenting device wherein the device is at least equivalent to distal or proximal protection in the bypass conduit intervention.

It is yet another advantage of the invention to provide a thrombectomy and balloon angioplasty/stenting device wherein the device provides simple thrombectomy by aspiration. Thrombectomy offers added benefits during coronary artery angioplasty and stenting in patients with ST-segment elevation myocardial infarction.

It is still another advantage of the invention to provide a thrombectomy and balloon angioplasty/stenting device with less induced ischemia-time than known proximal or distal balloon occlusion protection devices.

It is yet another advantage of the invention to provide a thrombectomy and balloon angioplasty/stenting device wherein the device does not add any significant delays in door to balloon time in patients with ST segment elevation myocardial infarction.

It is still another advantage of the invention to provide a thrombectomy and balloon angioplasty/stenting device wherein the device provides protection against distal embolization as the device offers similar or better outcome compared to catheter intervention with simple thrombectomy aspiration.

It is yet another advantage of the invention to provide a thrombectomy and balloon angioplasty/stenting device wherein the device is expected to decrease no-reflow phenomenon, with associated decrease in mortality and improved cardiac function after primary angioplasty and stenting.

It is still another advantage of the invention to provide a thrombectomy and balloon angioplasty/stenting device wherein the device includes added aspiration such that the device provides similar or better outcome to bypass catheter intervention than the conventional proximal or distal protection devices.

It is yet another advantage of the invention to provide a thrombectomy and balloon angioplasty/stenting device wherein the device is easier to use than the available devices such that more patients will receive bypass intervention with protection. This is expected to improve the clinical outcome of the procedure.

These and other features, aspects, and advantages of the present invention will become better understood upon consideration of the following detailed description, drawings, and appended claims.

### BRIEF DESCRIPTION OF THE DRAWINGS

FIG. 1 is a side view of one embodiment of a thrombectomy and balloon angioplasty device according to the invention including a proximal protection balloon.

FIG. 2 is a detailed cross-sectional view of the thrombectomy and balloon angioplasty device of FIG. 1 taken along line 2-2 of FIG. 1.

FIG. 3 is a detailed cross-sectional view, similar to FIG. 2, of another embodiment of a thrombectomy and balloon angioplasty device according to the invention.

Like reference numerals will be used to refer to like parts from Figure to Figure in the following description of the drawings.

### DETAILED DESCRIPTION OF THE INVENTION

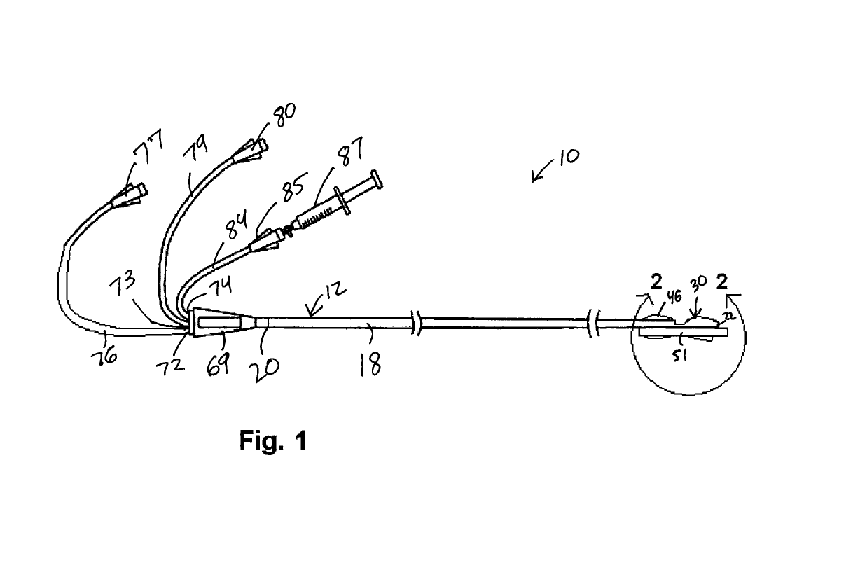

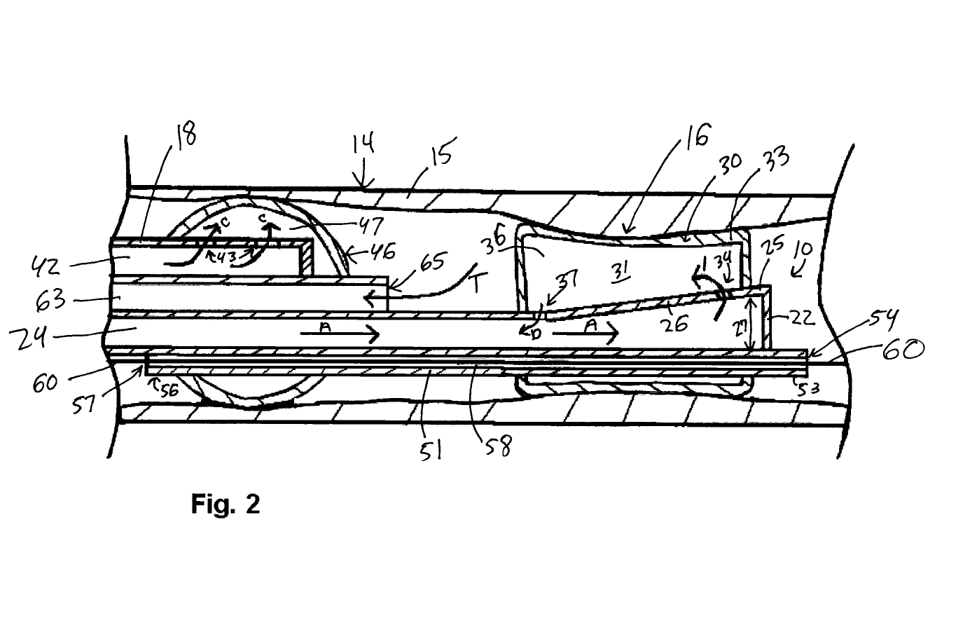

Looking FIGS. 1 and 2, there is shown one example embodiment of a thrombectomy and balloon angioplasty device 10 according to the invention. The thrombectomy and balloon angioplasty device 10 includes a catheter 12 for insertion in a blood vessel 14 having a wall 15. The wall 15 has a stenosis 16 formed from embolic material. The catheter 12 has a flexible shaft 18 having a proximal end 20 and a distal end 22. The shaft 18 has an angioplasty balloon inflation lumen 24 having a distal end 25. The angioplasty balloon inflation lumen 24 has an outwardly flaring wall 26 that creates a larger angioplasty balloon inflation lumen diameter 27 at the distal end 25 of the angioplasty balloon inflation lumen 24.

The shaft 18 can be formed from a polyurethane base polymer. Polyurethane can offer advantages over other materials as it is a more durable material enabling the use of thinner lumen walls. It offers less friction for ease of insertion; it is biocompatible; it has good tensile properties for safe insertion without kinks or fractures; it is resistant to hydrolysis, oxidation, oils and thermal degradation; it is thromboresistant and non-hemolytic; and it is rigid at room temperature but softer at body temperature to become more pliant, flexible and kink resistant when inserted into a blood vessel. In one example form, the shaft 18 is about 125-145 centimeters, and preferably is 135 centimeters in length. For peripheral vascular procedures, lengths for the shaft 18 ranging from 35 to 65 centimeters are useful.

The thrombectomy and balloon angioplasty device 10 includes a generally cylindrical angioplasty balloon 30 which is shown in an inflated condition engaging embolic material of the stenosis 16 in FIG. 2. The angioplasty balloon 30 defines an interior space 31 of the angioplasty balloon 30. A distal end 33 of the angioplasty balloon 30 is provided with inflation fluid from the angioplasty balloon inflation lumen 24 by way of inflation port 34. A one way check valve (such as a duckbill valve) is provided in the inflation port 34 such that fluid only flows from the angioplasty balloon inflation lumen 24 into the interior space 31 of the angioplasty balloon 30 as shown by arrow I in FIG. 2. A proximal end 36 of the angioplasty balloon 30 moves fluid from the interior space 31 of the angioplasty balloon 30 to the angioplasty balloon inflation lumen 24 by way of a deflation port 37. A one way check valve (such as a duckbill valve) is provided in the deflation port 37 such that fluid only flows from the interior space 31 of the angioplasty balloon 30 into angioplasty balloon inflation lumen 24 as shown by arrow D in FIG. 2.

The angioplasty balloon 30 can comprise a non-perforated low-compliance polymer such as polyethylene, polyethylene terephthalate, nylon or polyvinyl chloride. The angioplasty balloon 30 typically expands 5-30% when inflated to the rated pressure which may be 5-20 atmospheres. The angioplasty balloon 30 diameter is made in different sizes for each vessel family. For example, for percutaneous transluminal coronary angioplasty, the angioplasty balloon 30 may be 1-5 millimeters in diameter when inflated and 5-40 millimeters in length and used at pressures of 10-20 atmospheres. For percutaneous transluminal angioplasty, the angioplasty balloon 30 may be 4-12 millimeters in diameter when inflated, 20-100 millimeters in length, and used at pressures of 8-20 atmospheres.

The shaft 18 of the thrombectomy and balloon angioplasty device 10 includes an occlusion balloon inflation lumen 42 that provides fluid by way of inflation ports 43 to a generally spherical occlusion balloon 46 having an interior space 47. The occlusion balloon 46 can comprise a non-perforated compliant polymer such as polyurethane, latex or silicone. The occlusion balloon 46 can typically expand up to 25 millimeters diameter when inflated. Typically, the occlusion balloon 46 expands 100-600% when inflated to a rated pressure of less than 5 atmospheres. In another version of the invention, the device does not include an occlusion balloon for proximal protection. In other words, the invention can work with and without a proximal protection balloon.

The thrombectomy and balloon angioplasty device 10 includes a guide wire lumen 51 having a distal end 53 that terminates in a distal opening 54. The guide wire lumen 51 has a proximal end 56 that terminates in a proximal opening 57 proximal to the occlusion balloon 46. The guide wire lumen 51 is attached to the distal end 22 of the shaft 18 as shown in FIG. 2. A generally cylindrical interior space 58 of the guide wire lumen 51 is dimensioned to receive a guide wire 60 in a sliding and preferably sealing relationship, or near sealing relationship to provide a very low blood flow around the wire to facilitate aspiration of thromboembolic material. An example guide wire 60 is about 165 centimeters long and has a 0.014″ diameter. The guide wire lumen 51 can be up to about 200 millimeters long, with about 100 millimeters long being typical.

The shaft 18 of the thrombectomy and balloon angioplasty device 10 also includes a thrombectomy lumen 63 having a thrombectomy suction port 65 at its distal end.

Looking at FIG. 1, the thrombectomy and balloon angioplasty device 10 includes a catheter manifold 69 having an angioplasty balloon inflation fluid supply port 72, an occlusion balloon inflation fluid supply port 73, and a thrombectomy exit port 74. The angioplasty balloon inflation fluid supply port 72 is in fluid communication with the angioplasty balloon inflation lumen 24 and a catheter extension tube 76 that terminates in a connector 77 that may be connected to a source of angioplasty balloon inflation fluid (not shown). The occlusion balloon inflation fluid supply port 73 is in fluid communication with the occlusion balloon inflation lumen 42 and a catheter extension tube 79 that terminates in a connector 80 that may be connected to a source of occlusion balloon inflation fluid (not shown). The thrombectomy exit port 74 is in fluid communication with the thrombectomy lumen 63 and a catheter extension tube 84 that terminates in a connector 85 that is connected to a syringe 87 that allows for a suction force to be transmitted from the syringe 87 through the connector 85, the catheter extension tube 84, the thrombectomy exit port 74, the catheter manifold 69, the thrombectomy lumen 63 and the thrombectomy suction port 65. Other instruments for creating a suction force can also be connected to the connector 85.

Having described the structure of the thrombectomy and balloon angioplasty device 10, an exemplary use of the device 10 can be explained in further detail. The physician will use a guide catheter to engage the main blood vessel. The physician threads the guide wire 60 into a patient's blood vessel, moves the guide wire 60 against the blood flow in the artery, and then eventually advances the distal end of the guide wire with the blood flow in a blood vessel 14 beyond a stenosis 16 in the blood vessel 14 as in FIG. 2. Non-limiting examples of the blood vessel 14 having a stenosis include veins, and coronary arteries, subclavian arteries, the brachiocephalic artery, carotid arteries, renal arteries, and arteries in the legs. The distal opening 54 of the guide wire lumen 51 is then threaded onto the end of the guide wire 60 outside the patient's body. The guide wire lumen 51 is advanced on the guide wire 60 until the angioplasty balloon 30 is located in the blood vessel 14 adjacent the stenosis 16. The occlusion balloon 46 is then inflated by providing an inflation fluid from a source of occlusion balloon inflation fluid through the connector 80, the occlusion balloon inflation fluid supply port 78, the occlusion balloon inflation lumen 42, the inflation ports 43 and into the interior space 47 of the occlusion balloon 46 as shown in arrows C in FIG. 2 such the occlusion balloon 46 occludes the blood vessel 14 as shown in FIG. 2. As a result, blood does not flow distally beyond the occlusion balloon 46.

Aspiration can then be performed by applying a suction force from syringe 87 through the connector 85, the catheter extension tube 84, the thrombectomy exit port 74, the catheter manifold 69, the thrombectomy lumen 63 and the thrombectomy suction port 65. Embolic material and clot debris enters the thrombectomy suction port 65 as shown by arrow T in FIG. 2. The aspiration can be performed as a first step, or the aspiration can be performed at any time or continuously.

Then the angioplasty balloon 30 is inflated to eliminate the stenosis 16 by providing an inflation fluid from a source of angioplasty balloon inflation fluid through the connector 77, the angioplasty balloon inflation fluid supply port 72, the angioplasty balloon inflation lumen 24, the inflation port 34 and into the interior space 31 of the angioplasty balloon 30. See arrows A and arrow I in FIG. 2.

More aspiration is then performed by applying a suction force from syringe 87 through the connector 85, the catheter extension tube 84, the thrombectomy exit port 74, the catheter manifold 69, the thrombectomy lumen 63 and the thrombectomy suction port 65. Additional embolic material and clot debris enters the thrombectomy suction port 65 as shown by arrow T in FIG. 2. The angioplasty balloon 30 and the occlusion balloon 46 are then deflated and antigrade flow is resumed with no embolic material and clot debris traveling downstream in the blood vessel 14.

Optionally, an unexpanded stent can be slipped over the deflated angioplasty balloon 30 before the catheter 12 is threaded into the blood vessel. When the physician has inserted the catheter 12 to the site of the stenosis 16, the angioplasty balloon 30 is inflated, causing the stent to expand. The physician then removes the angioplasty balloon 30, leaving the expanded stent in place and allowing blood to once again flow freely through the blood vessel 14.

Advantageously, the angioplasty balloon 30 and the angioplasty balloon inflation lumen 24 are structured such that a distal diameter near the distal end 33 of the angioplasty balloon 30 reaches its maximum dimension in the blood vessel 14 before a proximal diameter near proximal end 36 of the angioplasty balloon 30 reaches its maximum dimension in the blood vessel 14 during inflation of the angioplasty balloon 30. Looking at FIG. 2, an example means for inflating the angioplasty balloon 30 in this manner is shown. When inflation fluid is injected into the angioplasty balloon inflation lumen 24, fluid only flows from the angioplasty balloon inflation lumen 24 into the interior space 31 of the angioplasty balloon 30 through inflation port 34 as shown by arrow I in FIG. 2 because of the one way check valve provided in the inflation port 34 and the one way check valve provided in the deflation port 37. As a result, fluid enters the distal portion of the interior space 31 of the angioplasty balloon 30 first thereby taking the distal portion of the interior space 31 of the angioplasty balloon 30 to its maximum dimension in the blood vessel 14 first. Thereafter, further injection of inflation fluid in to the interior space 31 of the angioplasty balloon 30 takes the proximal portion of the interior space 31 of the angioplasty balloon 30 to its maximum dimension in the blood vessel 14. In addition, the flaring wall 26 creates the larger angioplasty balloon inflation lumen diameter 27 at the distal end 25 of the angioplasty balloon inflation lumen 24 such that less fluid is needed to cause the distal portion of the interior space 31 of the angioplasty balloon 30 to reach its maximum dimension in the blood vessel 14. This is one example version of the angioplasty balloon 30; however, the final balloon can be cylindrical in addition to conical. The balloon could also be made from more compliant material in its distal end making the distal end inflate first before the proximal end and deflate last after the proximal end with or without one way check valves.

When the angioplasty balloon 30 is deflated by suctioning inflation fluid from the angioplasty balloon inflation lumen 24, fluid only flows from the interior space 31 of the angioplasty balloon 30 through deflation port 37 as shown by arrow D in FIG. 2 because of the one way check valve provided in the deflation port 37 and the one way check valve provided in the inflation port 34. As a result, fluid exits the proximal portion of the interior space 31 of the angioplasty balloon 30 first thereby taking the proximal portion of the interior space 31 of the angioplasty balloon 30 to its relaxed deflated dimension first. Thereafter, further section of inflation fluid from the interior space 31 of the angioplasty balloon 30 takes the distal portion of the interior space 31 of the angioplasty balloon 30 to its relaxed deflated dimension.

Therefore, the angioplasty balloon 30 and the angioplasty balloon inflation lumen 24 can be structured such that the angioplasty balloon 30 inflates gradually and not cylindrically, i.e., the angioplasty balloon 30 will start inflating from the distal end 33 to the proximal end 36; and stay at equal or larger diameter at the distal end 33 compared to the proximal end 36. This squeezes the embolic and clot debris back into the thrombectomy suction port 65 as shown by arrow T in FIG. 2. When the angioplasty balloon 30 is deflated; it will deflate from the proximal end 36 to the distal end 33 allowing the aspiration of the debris before the distal end 33 is fully deflated such that the distal end 33 of the angioplasty balloon 30 functions like a distal protection balloon.

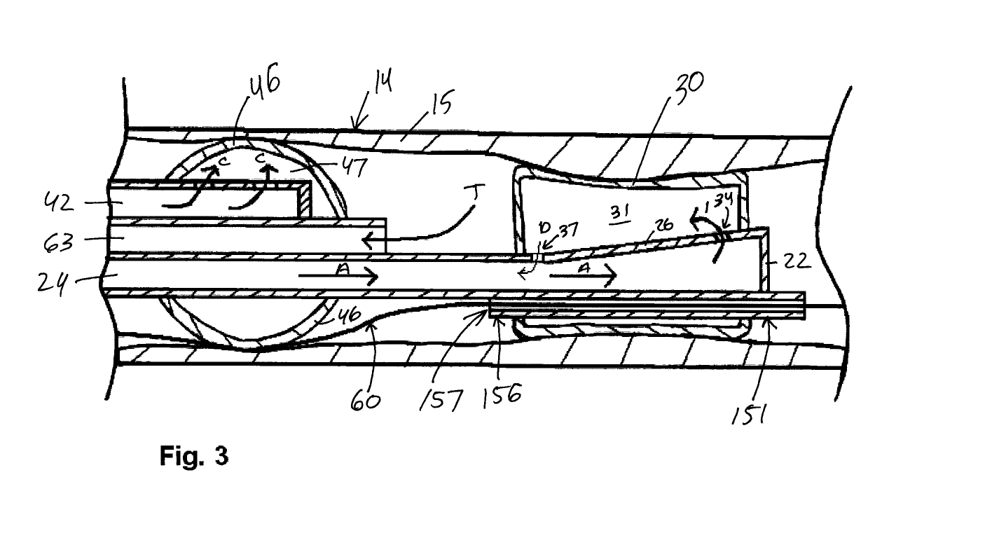

Turning to FIG. 3, there is shown another embodiment of a thrombectomy and balloon angioplasty device 110 according to the invention. The thrombectomy and balloon angioplasty device 110 is similar to the thrombectomy and balloon angioplasty device 10 of FIGS. 1 and 2. However, in the thrombectomy and balloon angioplasty device 110, the guide wire lumen 151 has a proximal end 156 that terminates in a proximal opening 157 distal to the occlusion balloon 46.

Thus, the invention provides a thrombectomy and balloon angioplasty/stenting device wherein the device combines protection, thrombectomy, and therapy in one easy to use device.

Although the present invention has been described in detail with reference to certain embodiments, one skilled in the art will appreciate that the present invention can be practiced by other than the described embodiments, which have been presented for purposes of illustration and not of limitation. Therefore, the scope of the appended claims should not be limited to the description of the embodiments contained herein.

What is claimed is:

1. A method for coronary angioplasty, the method comprising:
(a) threading a guide wire into a patient's coronary artery;
(b) advancing a thrombectomy and balloon angioplasty catheter on the guide wire until the thrombectomy and balloon angioplasty is located in the coronary artery adjacent a stenosis, the thrombectomy and balloon angioplasty catheter comprising: (i) a shaft having a proximal end and a distal end; (ii) an angioplasty balloon mounted to the shaft adjacent the distal end of the shaft; (iii) an occlusion balloon mounted to the shaft at a location proximally spaced from the angioplasty balloon; and (iv) a guide wire lumen arranged on the shaft for receiving the guide wire, the guide wire lumen extending from a first position adjacent the distal end of the shaft to a second position proximal the angioplasty balloon and distal the proximal end of the shaft, wherein the shaft includes an angioplasty balloon inflation lumen in fluid communication with the proximal end of the shaft and an interior space of the angioplasty balloon, an occlusion balloon inflation lumen in fluid communication with the proximal end of the shaft and an interior space of the occlusion balloon, and a thrombectomy lumen in fluid communication with the proximal end of the shaft and a thrombectomy suction port located between the angioplasty balloon and the occlusion balloon, wherein the angioplasty balloon inflation lumen and the guide wire lumen are not coaxial, and wherein the angioplasty balloon is non-perforated, and wherein the catheter is structured such that that blood does not flow distally beyond the occlusion balloon, and wherein the occlusion balloon is a sole occlusion balloon of the catheter;
(c) inflating the occlusion balloon;
(d) applying a suction force to the thrombectomy suction port; and
(e) inflating the angioplasty balloon to eliminate the stenosis.

2. The method of claim 1 further comprising:
(f) applying a second suction force to the thrombectomy suction port.

3. The method of claim 2 further comprising:
(g) deflating the occlusion balloon.

4. The method of claim 1 wherein:
the second position is proximal the occlusion balloon.

5. The method of claim 1 wherein:
the second position is distal the occlusion balloon.

6. The method of claim 1 wherein:
no balloons are located distal the angioplasty balloon.

7. The method of claim 1 wherein the catheter further comprises:
means for inflating the angioplasty balloon such that a distal diameter of the angioplasty balloon reaches its maximum dimension before a proximal diameter of the angioplasty balloon reaches its maximum dimension.

8. The method of claim 7 wherein:
the means for inflating the angioplasty balloon comprises an inflation port in fluid communication with the angioplasty balloon inflation lumen, the inflation port being located adjacent a distal end of the interior space of the angioplasty balloon.

9. The method of claim 1 wherein the catheter further comprises
means for deflating the angioplasty balloon such that the distal diameter of the angioplasty balloon reaches its relaxed position after the proximal diameter of the angioplasty balloon reaches its relaxed position.

10. The method of claim 9 wherein:
the means for deflating the angioplasty balloon comprises a deflation port in fluid communication with the angioplasty balloon inflation lumen, the deflation port being located adjacent a proximal end of the interior space of the angioplasty balloon.

11. The method of claim 1 wherein:
the occlusion balloon is non-perforated.

12. The method of claim 1 further comprising:
arranging a stent over the angioplasty balloon before inflating the angioplasty balloon; and
expanding the stent by inflating the angioplasty balloon.

13. The method of claim 1 wherein:
the angioplasty balloon is 1-5 millimeters in diameter when inflated.

14. A coronary angioplasty thrombectomy and balloon angioplasty catheter, the catheter comprising:
a shaft having a proximal end and a distal end;
an angioplasty balloon mounted to the shaft adjacent the distal end of the shaft;
an occlusion balloon mounted to the shaft at a location proximally spaced from the angioplasty balloon; and
a guide wire lumen arranged on the shaft, the guide wire lumen extending from a first position adjacent the distal end of the shaft to a second position proximal the angioplasty balloon and distal the proximal end of the shaft,
wherein the shaft includes an angioplasty balloon inflation lumen in fluid communication with the proximal end of the shaft and an interior space of the angioplasty balloon, an occlusion balloon inflation lumen in fluid communication with the proximal end of the shaft and an interior space of the occlusion balloon, and a thrombectomy lumen in fluid communication with the proximal end of the shaft and a thrombectomy suction port located between the angioplasty balloon and the occlusion balloon, and
wherein the angioplasty balloon inflation lumen and the guide wire lumen are not coaxial, and
wherein the angioplasty balloon is non-perforated, and
wherein the catheter is structured such that that blood does not flow distally beyond the occlusion balloon, and
wherein the occlusion balloon is a sole occlusion balloon of the catheter.

15. The catheter of claim 14 wherein:
the second position is proximal the occlusion balloon.

16. The catheter of claim 14 wherein:
the second position is distal the occlusion balloon.

17. The catheter of claim 14 wherein:
no balloons are located distal the angioplasty balloon.

18. The catheter of claim 14 further comprising:
means for inflating the angioplasty balloon such that a distal diameter of the angioplasty balloon reaches its maximum dimension before a proximal diameter of the angioplasty balloon reaches its maximum dimension.

19. The catheter of claim 14 wherein:
the occlusion balloon is non-perforated.

20. The catheter of claim 14 further comprising:
an expandable stent arranged over the angioplasty balloon.
